(19) United States
(12) Patent Application Publication
(10) Pub. No.: US 2012/0010567 A1
(43) Pub. Date: Jan. 12, 2012

(54) SINGLE USE SELF-DISABLING SYRINGE

(75) Inventor: Zou Qian Lin, Zhejiang Provence (CN)

(73) Assignee: GLOBAL MEDISAFE HOLDINGS LTD, Newcastle, NSW (AU)

(21) Appl. No.: 12/377,631

(22) PCT Filed: Aug. 14, 2007
(86) PCT No.: PCT/AU07/01138

§ 371 Date: Oct. 3, 2011

(30) Foreign Application Priority Data
Aug. 14, 2006 (CN) 200620106899.6

(51) Int. Cl. A61M 5/50

(52) U.S. Cl. 604/110

(57) ABSTRACT

A single use self-disabling syringe is disclosed, which includes a barrel, a plunger, and a rubber seal. At the end of the barrel there is a needle set. The front end the plunger has a clip, which can completely block the needle set. The syringe has the said needle set and barrel as a one-piece moulded, integrated unit. The said barrel has one supporting body at the front end, the supporting body and the inside wall of barrel fitting tightly. The said supporting body has a flexible hook on its top. The said clip and the hook have corresponding cone steps to make sure they match and seal.

### TECHNICAL FIELD

This invention relates to a syringe. In particular, it relates to a single use, self-disabling syringe.

### BACKGROUND

Syringes are a necessary implement in the medical field. Throughout the world, the multiple use of hypodermic syringe products that are intended for single use only is instrumental in drug abuse and more particularly in the transfer of contagious diseases. Intravenous drug users who routinely share and reuse syringes are a high risk group with respect to the AIDS virus. Also, the effects of multiple use are a major concern in third world countries where repeated use of syringe products may be responsible for the spread of many diseases. Reuse of single use syringe, assemblies is also instrumental in the spread of drug abuse even in the absence of infection disease.

Many attempts have been made to remedy this problem. Some of these attempts have required a specific act to destroy the syringe after use either by using a destructive device or providing a syringe assembly with frangible zones so that the syringe could be rendered inoperable by the application of force. Furthermore with the high cost of production it is not easy to produce in a high quantity.

### OBJECT OF THE INVENTION

It is therefore an object of the present invention to provide a unique and new invention and design for a single use self-disabling syringe which solves at least one major problem, namely to provide a single use and self-disabling syringe, where the construction is reasonably simple, the development of the moulds and assembling is easy and the syringe is completely destroyed after use.

At the very least, the invention provides an alternate single use syringes.

### DISCLOSURE OF THE INVENTION

According to the present invention, there is provided a single use self-disabling syringe, which includes a barrel, a plunger, and a rubber seal located on the plunger and providing a seal between the plunger and the barrel, wherein at the end of the barrel there is a needle set, and at the front end of the plunger there is a clip, such that the clip can completely block the needle set, the syringe characterised in that the said needle set and barrel is a one-piece moulded, integrated unit; the said barrel has a supporting body at the front end, wherein the supporting body and the inside wall of the barrel fit tightly together; the said supporting body having a flexible hook on its top; and the said clip has a cone step to engage with the flexible hook to make sure they match and seal.

Preferably, the hook has two or more flexible metal arms to assist in engaging with the clip. According to the above embodiment, it will be understood that this one-piece construction of the needle set and barrel lowers the production cost; the flexible metal arms and the clip hooked up tight and strong, such that when the plunger is pulled back there is no opportunity of the clip of the plunger disengaging from the hook; and the structure for this supporting body is simple and much easier to assemble.

In order to solve the technical issues, this practical new invention has taken on other embodiments. The said barrel has a supporting body at the front end, the supporting body and the inside wall of barrel working perfectly close for a tight fit; this designed structure makes the supporting body and the barrel easier to assemble and stable. The said top of the barrel is provided with a concave groove and a matching convex ridge is provided on the outside bottom end of the supporting body which fit tightly against each other at the top end of the barrel. This structure makes the function of the supporting body more reliable.

The said clip and the plunger are moulded in one piece, which simplifies the production technology of the syringe and decreases the cost.

The said plunger is provided with several easy-to-break points. These points make sure that when the plunger is pulled back, the plunger will break and be self-disabled at the line along the easy-to-break points, thereby achieving the goal of completely self-disabling the syringe.

Preferably there is also provided a stopper on the bottom, ie free end, of the plunger. Accordingly there is a specially designed passing hole at the bottom part of the plunger to accommodate the stopper. There are two easy-to-break points along a line and the stopper combines as one piece with a point on bottom of the plunger, the stopper moving along with the point. Two inside walls of the said passing hole have a vein on each side, and the veins are elevated. The stopper thus avoids any accident that might destroy the syringe itself during transport and prior to use, ie before drawing the fluid.

According to another embodiment of the invention, there is provided a single use self-disabling syringe including a hollow barrel, a plunger and a rubber seal which sits on the top end of the plunger, the top end of the barrel being a one-piece moulded needle set, the top end of the plunger having a clip associated therewith, so the clip can block the inside of the said needle set, the syringe being characterised by having the said needle set and barrel as a one-piece moulded unit; the top end of the barrel and the needle set having a circle of convex steps; the said clip having a flexible metal component, and at the top of the metal component there are flexible metal arms; the said clip and the flexible metal arms having complementary cone shapes so as to match.

By this means, this one-piece needle set and barrel lowers the cost; the flexible metal arms and the clip tightly hold, so that when the plunger is pulled back it doesn't disengage; and the simple structure makes assembly even easier.

In order to solve the technical problems, there are further embodiments.

Accordingly, there is a single use self-disabling syringe for which the metal component is a hollow circular cone, and the flexible metal arms extend in two directions. This structure makes the flexible metal arms easier to pass through the cone step provided at the junction between the top end of the barrel with the needle set.

Preferably, this is a single use self-disabling syringe which has the feature whereby the said clip and the plunger can be separated, when the injection is finished, by pulling the plunger back with extra force, so as to make the plunger and the clip separate. The rubber seal also comes off, causing the clip to fall inside the barrel so that it won't come out, thereby achieving complete self disabling of the syringe.

Although the following description generally refers to a syringe of conventional size, no such limitation is intended thereby, and reference to a syringe is meant to encompass any other needle/syringe combination or needle alone including slimline syringes, where, by suitable adaptation, the invention may also be usefully applied.

### BRIEF DESCRIPTION OF THE DRAWINGS

The invention may be better understood from the following non-limiting description of preferred embodiments, in which:

FIG. 1 is a cross-sectional side view of a syringe according to a first embodiment of the invention showing the syringe prior to use;

FIG. 2 is a cross-sectional side view of the syringe of FIG. 1, when the syringe has finished drawing the fluid;

FIG. 3 is a cross-sectional side view of the syringe of FIG. 1, after the syringe has finished injection and in the first step of self-disabling;

FIG. 4 is a cross-sectional side view of the syringe of FIG. 1, when the self-disabling state is completed;

FIG. 5 is a cross-sectional side view of the barrel of the syringe of FIG. 1;

FIG. 6 is a cross-sectional side view of the supporting body of the syringe of FIG. 1.

FIG. 7 is a cross-sectional plan view of the supporting body of FIG. 6;

FIG. 8 is a cross-sectional side view of the plunger of the syringe of FIG. 1;

FIG. 9 is a cross sectional plan view of the plunger of FIG. 8 when viewed from the direction shown as A to A in FIG. 8;

FIG. 10 is a further cross sectional side view of the plunger of FIG. 8 when viewed from the direction shown as K in FIG. 8;

FIG. 11 is a partial magnified portion of the FIG. 1;

FIG. 12 is a cross-sectional side view of a syringe according to a second embodiment prior to use;

FIG. 13 is a cross-sectional side view of the syringe of FIG. 12 when the syringe has finished drawing the fluid;

FIG. 14 is a cross-sectional side view of the syringe of FIG. 12 after the syringe has finished injection and in the first step of self-disabling;

FIG. 15 is a cross-sectional side view the syringe of FIG. 12 when the self-disabling state is completed;

FIG. 16 is a cross-sectional side view of the barrel of the syringe of FIG. 12;

FIG. 17 is a cross-sectional side view of the plunger and the clip of the syringe of FIG. 12;

FIG. 18 is a perspective view of the flexible metal arms of the syringe of FIG. 12;

FIG. 19 is a partial magnified portion of FIG. 12.

### DETAILED DESCRIPTION OF THE DRAWINGS

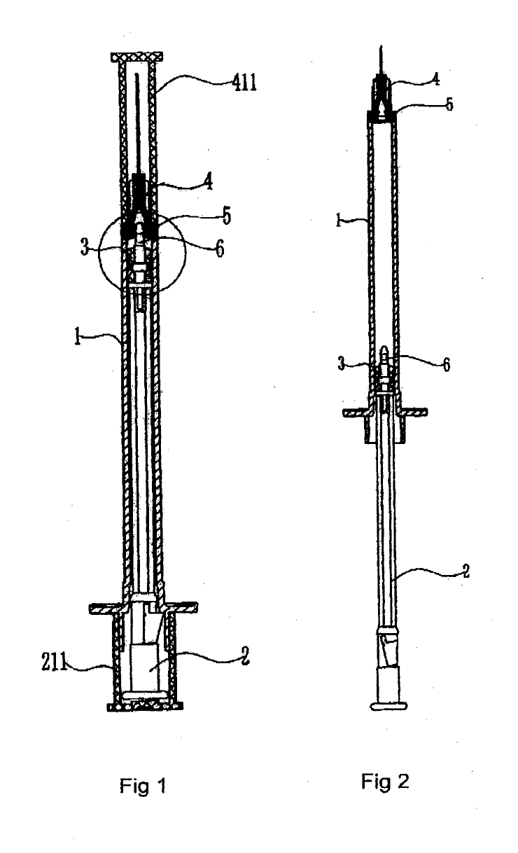

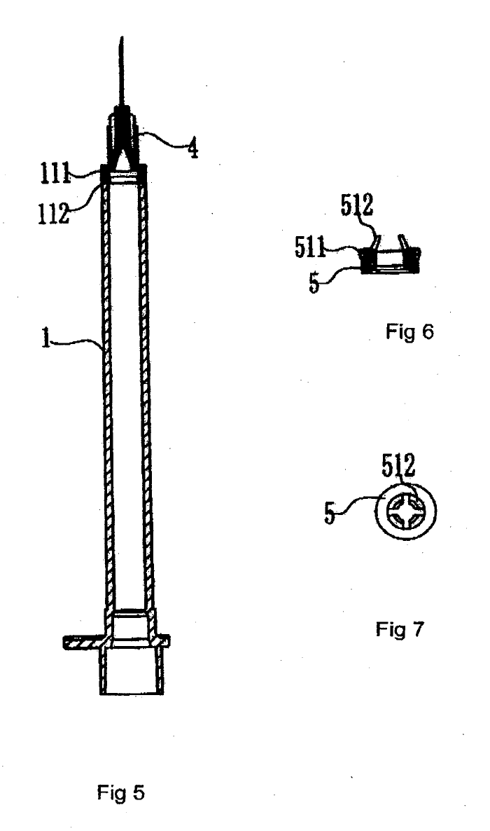

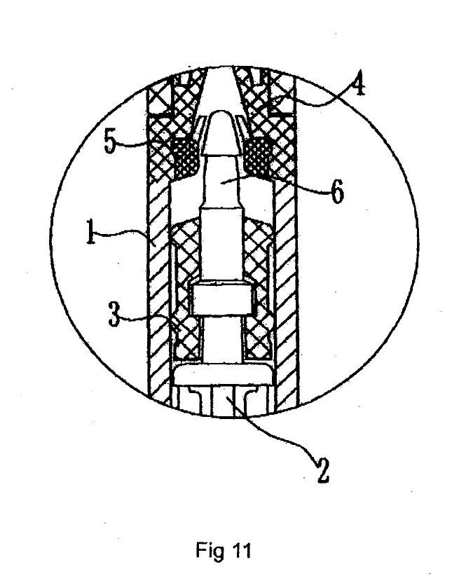

Referring generally to FIGS. 1 to 11, there is shown a first embodiment of the invention, being a single use self-destructing syringe including a barrel (1), a plunger (2), a rubber seal (3). At the end of the barrel (1) there is a needle set (4). At the front end the plunger (2) there is a clip (6), which can completely block the needle set (4), as shown in FIG. 4. The said barrel (1) has one supporting body (5) at the front end, the supporting body (5) and the inside wall of barrel (1) fitting tightly. The said supporting body (5) has a flexible hook comprising at least two fingers (512) on its top, as shown in FIGS. 6 and 7. The supporting body (5), which is preferably metal, is placed at the top end of the barrel (1).

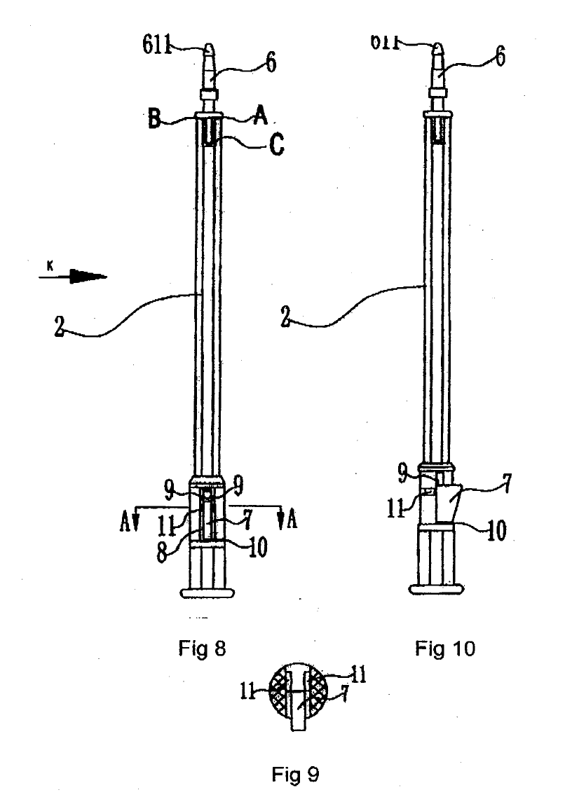

Referring to FIGS. 8 to 10, there is shown the clip (6) which is set at the top end of the plunger (2), and this is a one-piece moulded unit. There is a pointy step (611) that can easily go through the flexible metal arms (512), so that when the plunger (2) pushes the fluid during an injection, the clip (6) goes into the flexible arms (512), and then finally the flexible arms (512) stop or engage against at the base of the pointy step (611), so that the clip (6) can't pull back. In this way, the clip (6) can block the inside hole of needle set (4), so as to finish the first step if self-disabling.

There are several easy-to-break points on the plunger (2), and in this embodiment for example there are shown three points A, B, C in FIG. 8. When the injection is finished and the plunger (2) pulled back, pulling will cause the plunger to break at the points along the easy-to-break points, thereby disabling the plunger (2) against re-use again, therefore completely achieving the goal of self-disabling.

As stated before, this is a single use self-disabling syringe, and further special points are included. These include a stopper (7), and a specially designed passing hole (8) at the bottom part of the plunger (2) which accommodates the stopper (7). There are two easy to break points (9) in a line. These are combined as one piece with a point (10) on top of the plunger (2), the stopper (7) moving along with the point (10). Two inside walls of the said passing hole (8) each have a vein (11) on each side, and the veins are elevated. When the fluid is injected, it is only necessary to push gently toward the stopper (7) so as to make the stopper (7) move along the point (10), so as to cause the easy-to-break points (9) to break. This also stops the accidental self-disabling prior to use during transport and/or through any unnecessary mistakes.

Referring to FIGS. 1 to 4, the operation of this new practical invention is shown. In FIG. 1, there is shown the syringe prior to use, the clip (6) hasn't pass through the flexible hook (512), and in the meantime is prevented from mis-operation. In the following drawing FIG. 2, the syringe is shown when drawing of the fluid is finished, after the needle protection cap (411) and the protection lid (211) for the plunger have been removed. Also the outside surface of stopper (7) fits in the passing hole (8).

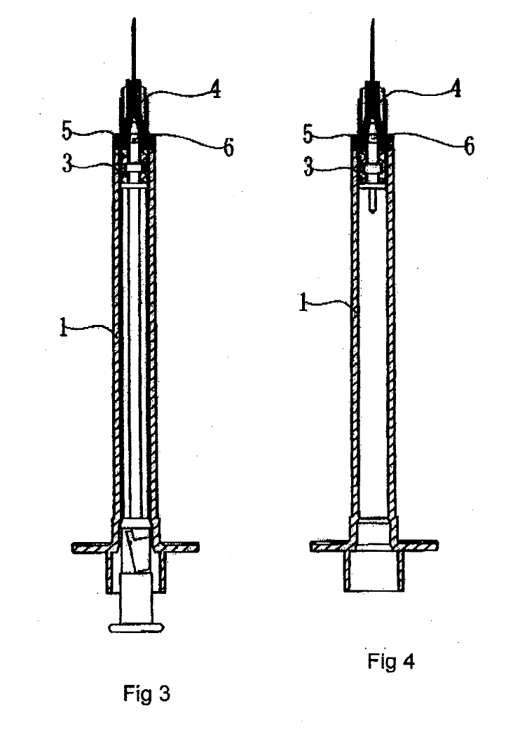

As per FIG. 3, when the injection is finished, the pointy step (611) of the said clip (6) will pass through the flexible metal hook (512) and this time, the hook (512) and the pointy step (611) click together and stop the clip (6) from moving, thereby blocking the inside surface of the needle set (4) by the clip (6), so that you couldn't reuse the syringe, thus finishing the first step of self-disabling. As shown in FIG. 4, there is shown the moment that self-disabling is completed. Pulling harder makes sure the plunger (2) is broken and the top end of the plunger breaks inside the barrel (1) but can't come out. This prevents the plunger (2) and rubber seal (3) from being changed so to prevent reuse again, and therefore the syringe is completely self-disabled.

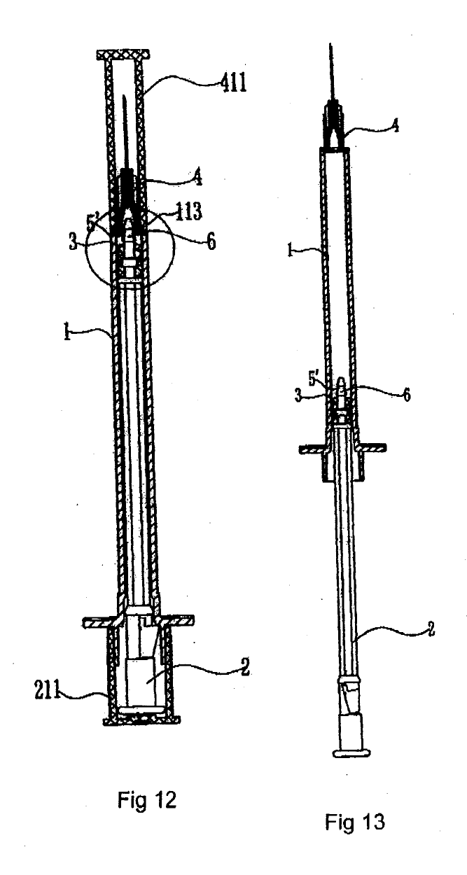

Referring generally to FIGS. 12 to 19, there is shown a second embodiment of the invention being a single use self-destructing syringe which includes a barrel (1), plunger (2), rubber seal (3), needle set (4), flexible metal hook (5′), clip (6), wherein the flexible metal hook (5′) sits on top of the clip (6).

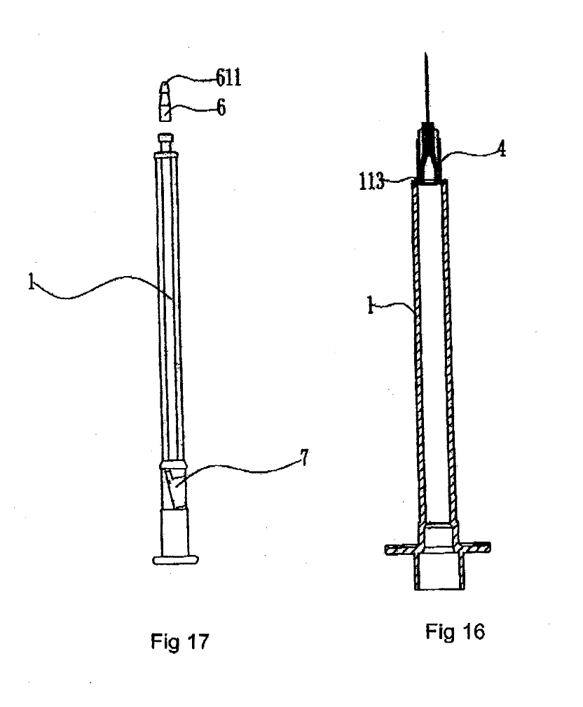

Viewing FIG. 16, the said needle set (4) and barrel (1) is a one-piece moulded, integrated unit. The said barrel (1) has a supporting body (5) at the front end, the supporting body (5) and the inside wall of barrel (1) fitting tightly. The said supporting body (5) has a flexible hook (512) on its top. The said clip (6) and the hook (512) have a pointy step (611) to make sure they match and seal properly. FIG. 17 shows the top end of the plunger (2) has one clip (6) and we previously indicated, they click together, but doesn't mean the needle is totally blocked. However, you only need to pull harder, and it will be apart from each other. A pointy step (611) is formed at the top end of the clip (6).

At the bottom end of the plunger (2), there is a designed stopper (7), the structure of the stopper (7) is same as the for the first embodiment shown in FIGS. 1 to 11 and functions in the same way.

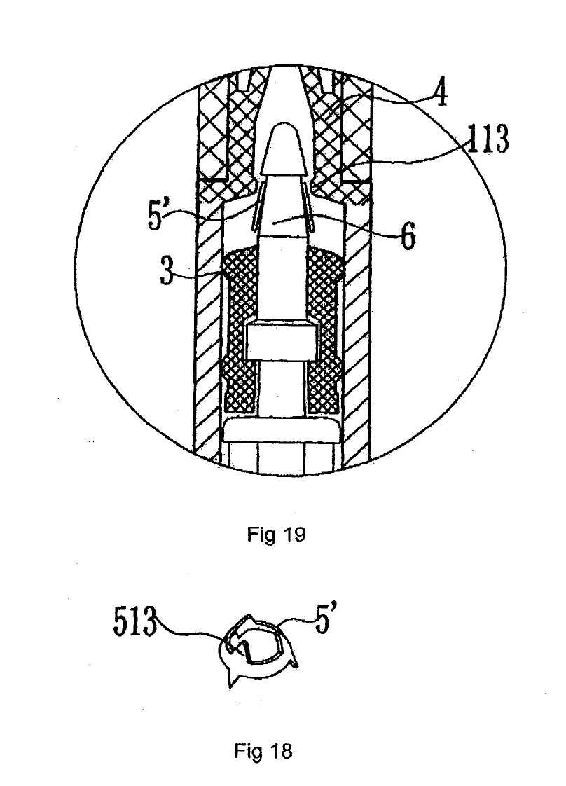

Referring to FIG. 18, the metal hook (5′) is made from metal and the main body is a hollow cone shaped part, and the top thereof is provided with flexible arms (513) with at least two such arms extending in two directions as shown, This flexible hook (513) is used so as to function in the same way as the previous embodiment where metal arms (512) were utilised. Once pushing the fluid is finished, the clip (6) passes through the step (113) that joins at the end of the barrel (1) with needle set (4). The end of this metal hook (513) is pushed against by the step (311), the top end is positioned by pointy step (611), so the clip (6) can't pull back again, and the clip (6) blocks the gateway of needle set (4).

Referring to FIGS. 12 to 15 there are shown the steps involved in using and disabling the syringe. Referring to FIG. 12, the syringe is shown prior to use, clip (6) hasn't passed through the convex step (113) of the joint between barrel (1) and the needle set (4), and also to avoid any accident or mis-use, the stopper (7) sticks against the bottom end of the barrel (1).

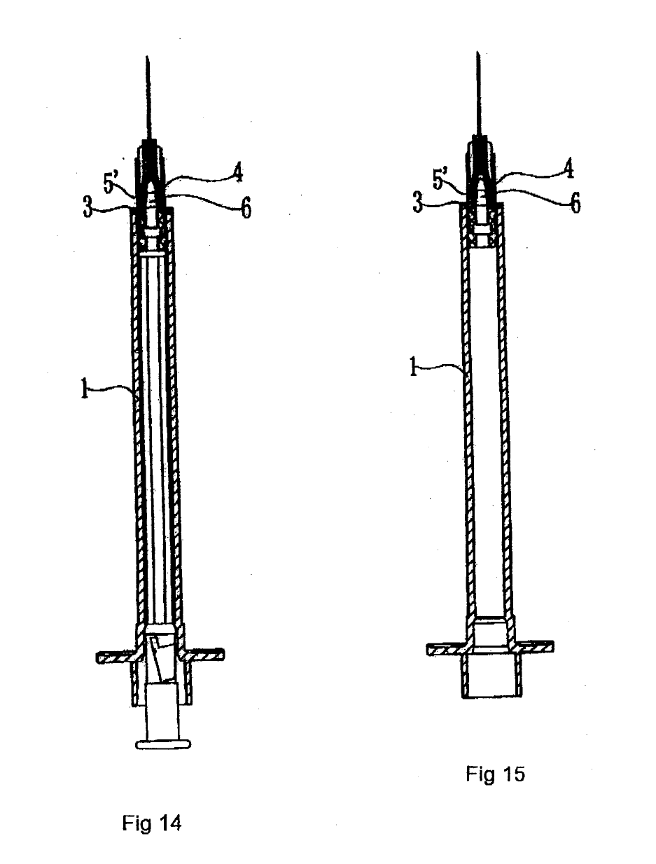

And then the following drawing, FIG. 13, shows the syringe when drawing the fluid has finished, at this stage, the needle cap (411) and plunger lid (211) both having been taken off, and the outside surface of stopper (7) fits in the passing hole (8). As per FIG. 14, when the injection is finished, the pointy step (611) of the mentioned clip (6) will pass through the flexible metal hook (512) and this time, the hook (512) and the pointy step (611) click together and stop the clip (6) from moving, thereby blocking the inside surface of the needle set (4) by the dip (6), so that the syringe cannot be reused, completing the first step of self-disabling.

As shown in FIG. 15, the syringe has finished the self-disabling, pulling harder having made sure the plunger (2) is broken. The top end of the plunger breaks inside the barrel (1) but can't come out, in order to avoid the plunger (2) and rubber seal (3) from being changed and thereby preventing the syringe from being reused again. Consequently, the syringe is completely self-disabled.

It will be appreciated by those skilled in the art that many modifications and variations may be made to the embodiments described herein without departing from the spirit or scope of the invention.

Throughout the specification the word “comprise” and its derivatives are intended to have an inclusive rather than exclusive meaning unless the context requires otherwise.

1. A single use self-disabling syringe, which includes a barrel, a plunger, and a rubber seal located on the plunger to provide a seal between the plunger and the barrel, wherein at the end of the barrel there is a needle set, and at the front end of the plunger there is a clip, such that the clip can completely block the needle set, the syringe characterised in that the said needle set and barrel is a one-piece moulded, integrated unit; the said barrel has a supporting body at the front end, wherein the supporting body and the inside wall of the barrel fit tightly together; the said supporting body having a flexible hook on its top; and the said clip has a cone step to engage with the flexible hook to make sure they match and seal.

2. A single use self-disabling syringe according to claim 1, wherein at the top end of the inside barrel, there is a groove of concave shape that matches a convex edge on the supporting body.

3. A single use self-disabling syringe according to claim 1, further characterised in that at the top of the barrel there is a concave groove and a matching convex ridge on the outside bottom end of the supporting body, there being a tight fit between the convex ridge with the top end of the barrel.

4. A single use self-disabling syringe according to claim 1, wherein the clip and the plunger are plastic and moulded in one piece.

5. A single use self-disabling syringe according to claim 1, wherein the said plunger is provided with several easy-to-break points.

6. A single use self-disabling syringe according to claim 1, wherein there is provided a stopper, and a corresponding passing hole at the bottom part of the plunger, to accommodate the stopper, there being at least two easy-to-break points along a line, the stopper combining as one piece with a point on top of the plunger, the stopper moving along with the point, and wherein two inside walls of the said mentioned passing hole have a vein on each side, the veins being elevated.

7. A single use self-disabling syringe, including a hollow barrel, a plunger and a rubber seal which sits on the front end of the plunger to provide a seal with the barrel, the top end of the barrel being a one-piece moulded arrangement incorporating a net needle set, the top end of the plunger having a clip, the clip able to block the inside of the said needle set, the syringe characterised in that the top end of the said barrel and the needle set have a circle of convex steps; the said clip having a flexible metal component or hook located thereon, where at the top of the metal component there is one or more flexible metal arms; and the said clip and the metal flexible arms have a pointy step to match.

8. A single use self-disabling syringe according to claim 7, wherein the metal component is a hollow circular cone, and the metal flexible arms extend in two directions.

9. A single use self-disabling syringe according to claim 7, wherein the said clip and the plunger can be separated.

10. A single use self-disabling syringe according to claim 7, wherein there is also provided a stopper located at the bottom of the plunger, co-operating with a specially designed passing hole at the bottom part of the plunger which accommodates the stopper; there are also provided two easy-to-break points in a line; the stopper combining as one piece with a point on the bottom of the plunger, the stopper moving along with the point; and two inside walls of the said passing hole having a vein on each side, the veins being elevated.

11. (canceled)
